(19) United States
(12) Patent Application Publication
(10) Pub. No.: US 2015/0080638 A1
(43) Pub. Date: Mar. 19, 2015

(54) Therapeutic Magnetic Pouch for Pet Collar

(71) Applicant: Deborah Kunnari, Butte, MT (US)

(72) Inventor: Deborah Kunnari, Butte, MT (US)

(21) Appl. No.: 14/488,567

(22) Filed: Sep. 17, 2014

Related U.S. Application Data
(60) Provisional application No. 61/878,937, filed on Sep. 17, 2013.

(51) Int. Cl. A61N 2/06; A01K 27/00

(52) U.S. Cl. CPC A61N 2/06; A01K 27/008
USPC 600/15

(57) ABSTRACT

Disclosed is a therapeutic magnetic pouch for a pet collar. The device includes a pouch having an interior volume in which magnets can be placed. The pouch includes a hook and loop fastener along the top and bottom edges thereof. The pouch wraps around a portion of an existing pet collar and secures in position by overlapping the hook and loop fasteners. The magnets are disposed so that the north-pole of the magnets faces the outer surface of the pouch, which directly contacts a pet's neck. The magnets induce electromagnetic currents to the pet's body, which can be beneficial for inflammatory processes in elbow joints and knees, as well as for arthritis. The device also accelerates the healing process of fractures, sprains, and strains. In this way, the present invention provides a holistic and cost-effective alternative for pet medical care, increasing the overall health of the pet.

### CROSS REFERENCE TO RELATED APPLICATION

This application claims the benefit of U.S. Provisional Application No. 61/878,937 filed on Sep. 17, 2013. The above identified patent application is herein incorporated by reference in its entirety to provide continuity of disclosure.

### BACKGROUND OF THE INVENTION

1. Field of the Invention

The present invention relates to a magnetic pouch for a pet collar. More specifically, the present invention pertains to a pouch with imbedded magnets therein. The pouch is constructed so that it is adapted to removably attach to an existing pet collar. When the pouch is placed on the pet collar, the magnetic field produced by the magnets provides therapeutic benefits to the pet wearing the collar.

Many pets suffer from arthritis, joint pain, and hip and back injuries, as well as other stress-inducing conditions, such as separation anxiety. To treat these stress-inducing conditions, most veterinarians prescribe medication for pets. Many pet owners, however, prefer a holistic and natural approach to treating their pets.

Magnetic therapy for pets is a popular alternative treatment method used increasingly more by pet owners. The opposite poles of a magnet have different effects on the pet's body. The metabolism of the cells that make up a tissue can be stimulated by induced electromagnetic currents. The north-pole generally has strengthening, stimulating qualities. It can be used to give energy and improve weak organs. The south-pole, on the other hand, withdraws energy. It can be used to sedate and relax, for pain relief, to reduce unwanted growths, swellings, to soothe inflammations and to arrest infections. The magnet does not have to directly touch the pet, but can be kept in a pouch or a pocket, with the specified pole facing the pet's body.

Magnets are generally applied to pets around a pet's neck via a magnetic collar. Conventional magnetic pet collars, however, are only available in specific sizes, and have limited adjustability, which requires a pet owner to purchase one that properly fits the pet. Additionally, conventional magnetic pet collars are generally designed to replace existing pet collars, and therefore cannot be used with existing pet collars. Therefore, a device that can be used with existing pet collars and that provides magnetic therapy to pets is desired.

The present invention provides a therapeutic magnetic pouch for a pet collar is provided. The pouch has an interior volume that holds a plurality of neodymium magnets in a substantially linear configuration. The pouch is substantially rectangular and has a hook and loop fastener attached along the top and bottom edges thereof. The pouch wraps around a portion of an existing pet collar and is secured in position by overlapping the hook and loop fasteners disposed on the top and bottom portions of the pouch. The magnets are disposed so that the north-pole faces the outer surface of the pouch. When the pouch is placed on the collar, the magnets help to relieve pain and stress experienced by the pet.

2. Description of the Prior Art

Devices have been disclosed in the prior art that relate to magnetic pet collars. These include devices that have been patented and published in patent application publications, and generally relate to collars that are composed of magnetic materials or collars that include imbedded magnets therein. Some of these devices disclose a tubular pouch that secures a plurality of magnets to a pet collar. Other devices disclose a metal pet collar having copper electroplating. These devices, however, do not disclose pouch with imbedded magnets, wherein the pouch may be removably attached to a pet collar. The foregoing is a list of devices deemed most relevant to the present disclosure, which are herein described for the purposes of highlighting and differentiating the unique aspects of the present invention, and further highlighting the drawbacks existing in the prior art.

Specifically, U.S. Pat. No. 6,286,463 to Sykes discloses a magnetic animal restraint. In one embodiment, the animal restraint is a collar that is composed of magnetic material, such as magnetic fabric. In another embodiment, the animal restraint is a collar with an elongated sleeve. The elongated sleeve includes a hollow interior, in which one or more magnets are placed. While the device of Sykes discloses an animal restraint that includes one or more imbedded magnets therein, the elongated sleeve cannot be removed from the animal restraint. In this regard, Sykes device is not suitable for use with existing pet collars. In contrast, the present invention discloses a pouch that is adapted to removably attach to an existing pet collar via hook and loop closure. As such, the present invention increases versatility to the user.

Similarly, U.S. Published Patent Application Number 2007/0204811 to Agee discloses a therapeutic pet collar that is composed of iron, nickel, or an alloy chain having thin copper electroplating. The iron, nickel, or alloy conducts magnetic fields. A resilient plug magnetic holder may be inserted into a link, twisted and locked in place to hold magnets inside a link of the chain. A plurality of magnets may be attached to the collar. The magnets are disposed so that the polarities of the magnets alternate. Unlike the present invention, Agee fails to disclose a removably attached pouch with imbedded magnets therein. Furthermore, the disposition of the magnets in Agee differ from the present invention. In this regard, the magnets in Agee are disposed so that the polarity of the magnets alternates. In contrast, the magnets of the present invention do not alternate. In this way, different poles of the magnets may be applied to the pet as needed.

U.S. Pat. No. 6,344,021 to Juster discloses an adhesive magnetic therapy patch that is placed directly on the skin of a person. The adhesive patch has a hypoallergenic adhesive on one side and at least one magnet disposed thereon. The adhesive patch may include one or more depressions therein to accommodate one or more magnets. The purpose and design of the Juster invention differ from the present invention. The device of Juster is an adhesive patch that is designed for use with humans. In contrast, the present invention provides a pouch with imbedded magnets for use with pet collars to provide magnetic therapy to pets.

U.S. Pat. No. 3,589,341 to Krebs provides an animal collar that includes a magnetic fastener which is smooth and free of projections when coupled. The collar includes two terminal ends having a magnet thereon. The magnets are disposed so that opposite poles of the magnets are engaged when the collar is secured around a pet's neck. As such, the magnets are not removably attached to the collar as the collar would not be functional without the magnetic fasteners. In contrast, the purpose of the present invention is to provide magnetic therapy to pets. As such, the magnets can be removably attached to the collar via a pouch in which the magnets are disposed.

U.S. Design Pat. No. D523999 to Olson discloses an animal collar with magnets embedded throughout it to help relieve an animal's joint pain and muscle aches. The collar may be composed of leather or nylon material. The collar comprises an internal volume that contains one or more magnets therein. Although Olson discloses an animal collar containing embedded magnets, Olson does not disclose magnets that are embedded in a pouch that can be removably attached to an existing pet collar. The present invention allows a pet owner to apply magnets to a pet as needed without requiring the pet owner to put on an additional or new pet collar. Similarly, the present invention eliminates the need to remove the pet collar from the pet when removing the magnets.

Finally, U.S. Published Patent Application Number 2002/0046715 to McCarville discloses a pet collar that reduces shedding in pets. The collar comprises at least one magnet wherein the north pole of each of the magnets are directed towards the neck of the pet. The magnetic field produced is sufficient to reduce shedding in pets. The magnets are disposed between the pet collar straps and are spaced apart at regular intervals. Unlike the present invention, the magnets are not removable from the pet collar. Furthermore, McCarville does not disclose that the magnets provide therapeutic benefits to the pet wearing the pet collar.

The devices disclosed in the prior art have several known drawbacks. These devices are limited in the fact that they cannot be used with existing pet collars. These devices are designed to be used in lieu of existing pet collars so that a pet owner must purchase a new collar that properly fits the pet. The present invention overcomes these limitations by disclosing a magnetic pouch that can be removably attached to a portion of an existing pet collar. In this way, the magnetic pouch can be attached or removed as necessary to provide an appropriate amount of magnetic therapy for pets. It is therefore submitted that the present invention is substantially divergent in design elements from the prior art, and consequently it is clear that there is a need in the art for an improvement to existing magnetic pet therapy devices. In this regard, the instant invention substantially fulfills these needs.

### SUMMARY OF THE INVENTION

In view of the foregoing disadvantages inherent in the known types of magnetic pet collars now present in the prior art, the present invention provides a new and improved therapeutic magnetic pouch for a pet collar wherein the same can be utilized for providing magnetic therapy to pets.

It is therefore an object of the invention to provide a new and improved therapeutic magnetic pouch for a pet collar that has all of the advantages of the prior art and none of the disadvantages.

Another object of the present invention is to provide a new and improved therapeutic magnetic pouch for a pet collar that may be used with new and existing pet collars.

Yet another object of the present invention is to provide a new and improved therapeutic magnetic pouch for a pet collar that provides a holistic and cost-effective alternative for pet medical care by providing magnetic therapy, thereby increasing the overall health of the pet.

Still yet another object of the present invention is to provide a new and improved therapeutic magnetic pouch for a pet collar that is beneficial for joint pains, arthritis, hip and back injuries, fractures, sprains, and strains, as well as for other stress-inducing conditions, such as separation anxiety.

A further object of the present invention is to provide a new and improved therapeutic magnetic pouch for a pet collar that provides effective control, removal, and treatment of ticks and fleas.

An additional object of the present invention is to provide a new and improved therapeutic magnetic pouch for a pet collar that includes a pouch with an interior volume that holds a plurality of magnets therein.

Yet another object of the present invention is to provide a new and improved therapeutic magnetic pouch for a pet collar that includes a plurality of imbedded magnets that are disposed so that the north-pole of each of the magnets faces the outer surface of the pouch.

Still yet another object of the present invention is to provide a new and improved therapeutic magnetic pouch for a pet collar that wraps around a portion of a pet collar on which it is placed.

Other objects, features, and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein the numeral annotations are provided throughout.

FIG. 1 shows a perspective view of the present invention in an open configuration.

FIG. 2 shows a perspective view of the present invention in a closed configuration.

FIG. 3 shows a view of the present invention as secured to a pet collar.

### DETAILED DESCRIPTION OF THE INVENTION

References are made herein to the attached drawings. Like reference numerals are used throughout the drawings to depict like or similar elements of the therapeutic magnetic pouch for a pet collar. For the purposes of presenting a brief and clear description of the present invention, the preferred embodiment will be discussed as used to provide magnetic therapy to pets. The figures are intended for representative purposes only and should not be considered to be limiting in any respect.

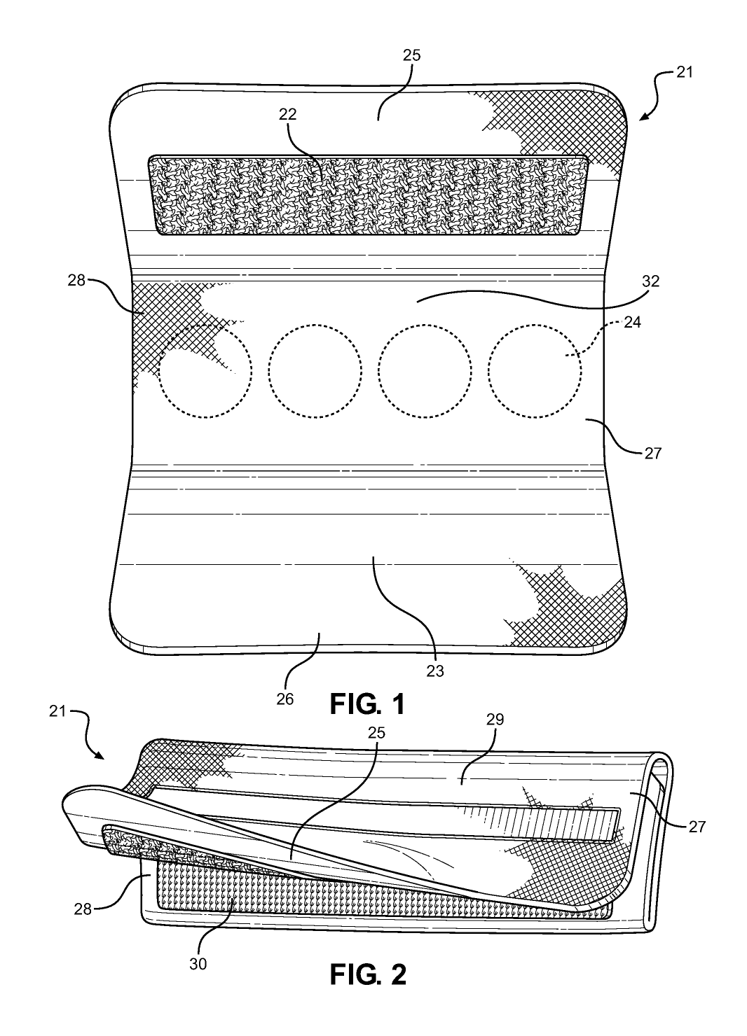

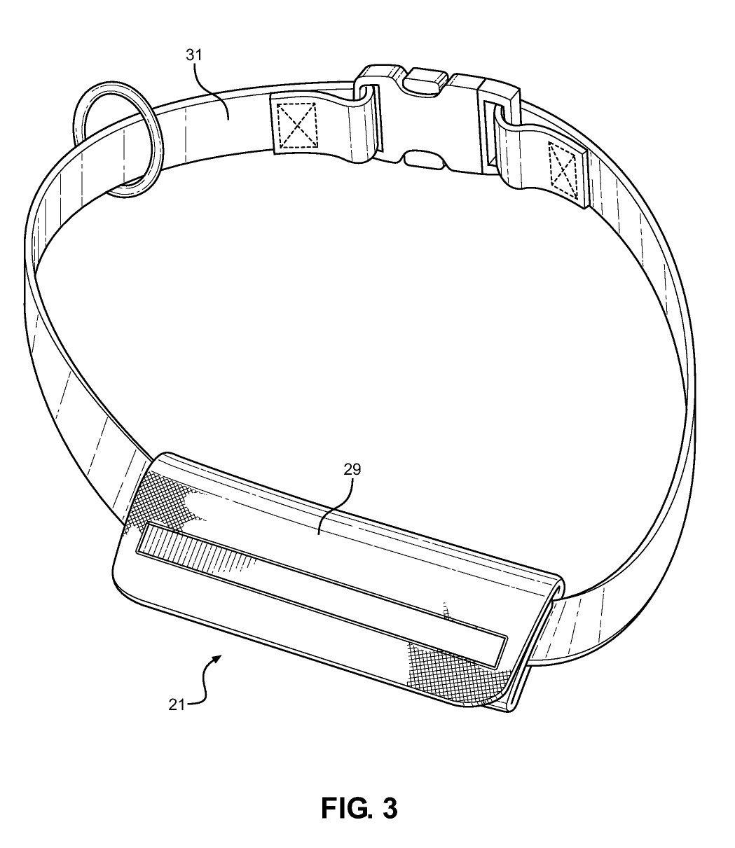

Referring now to FIGS. 1 and 2, there are shown perspective views of the present invention in an open configuration and a closed configuration, respectively. The present invention comprises a substantially rectangular pouch 21 having two sheets of juxtaposed fabric that are secured together to form an inner surface 23 and an outer surface 29. The two sheets of fabric may be composed of cotton, denim, polyester, or other suitable material. The sheets may also comprise various prints and designs thereon, and may further comprise ornamental piping, embellishments, or the like. The two juxtaposed sheets of fabric are stitched together in a straight line of stitches starting in proximity of one of their edges so as to form a top portion 25, a bottom portion 26, a first side 27, a second side 28, and an interior volume. Thereafter, the sheets are parallelly stitched together in horizontal rows of stitches to provide a central pocket 32 at substantial midpoint between the top 25 and bottom portions 26. In this way, the present pouch 21 provides three sections. In some embodiments, the pocket 32 may include padding therein to provide cushioning to a plurality of magnets 24 held therein.

The pouch 21 is folded at the horizontal rows so that the top portion 25 and the bottom portion 26 overlap one another. The inner surface 23 of the pouch 21 includes a hook fastener 22 near the top portion 25. The hook fastener 22 is adapted to engage a loop fastener 30 that is disposed on the outer surface 29 of the pouch 21 near the bottom portion 26. Alternatively, the loop fastener 30 may be disposed on the inner surface 23 near the top portion 25, and the hook fastener 22 may be disposed on the outer surface 29 near the bottom portion 26. In this way, the top 25 and bottom portions 26 are removably attached via hook and loop fasteners 22, 30. In other embodiments, the pouch 21 may include other fasteners such as snap fasteners, zippers, or the like.

The magnets 24 are inserted into the pocket 32 so that they are in a linear configuration. In a preferred embodiment, approximately four neodymium or ceramic magnet pieces may be used. Additionally, it is preferred that each of the magnets 24 has a magnetic field strength in the range of 500 to 5000 gauss. Neodymium magnets are the most widely used type of rare-earth magnet and are made from an alloy of neodymium, iron, and boron. Ceramic magnets, also known as ferrite magnets, are made of iron oxide and strontium carbonate. Each of the magnets 24 may be disk shaped. A first side of said disk shape corresponds to a north-pole and a second side corresponds to a south-pole of the magnet. The magnets 24 are positioned so that the north-pole of the magnets 24 faces the outer surface 29 and the south-pole of the magnets 24 faces the inner surface 23 of the pouch 21.

Referring now to FIG. 3, there is shown a view of the present invention as placed on a pet collar 31. The pouch 21 is dimensioned so that it is able to wrap around a portion of a pet collar. In use, a portion of the pet collar is placed adjacent to the inner surface of the pouch 21. Thereafter, the top and bottom portions of the pouch 21 are folded over the pet collar 31 and are secured together via the hook and loop fasteners thereon. In this way, the outer surface 29 of the pouch 21 directly contacts the pet when the pet collar 31 is worn around the pet's neck.

The magnets do not have to directly contact the pet's skin, but can be kept in the pouch with the north-pole facing the neck of the pet. When held against the pet's body, the magnets relax capillary walls, thereby boosting blood flow to the painful or injured area. The magnets also interfere with the electrochemical reactions that take place within nerve cells, impeding their ability to transmit pain messages to the brain. For general health improvement, magnets with stronger magnetic field strength may be worn for a few hours at a time, or magnets with weaker magnetic field strength may be worn all day. For serious infections, strong pain, or cancerous tumors, however, magnets with stronger magnetic field strength may remain on the body as long as they seem to be beneficial.

As discussed above, the present invention may be also provide effective control, removal, and treatment of ticks and fleas. In this regard, the magnets create a frequency barrier with the scalar waves produced by the combination of Earth's magnetic field and a movement of blood circulation. Scalar waves create an environment that parasites cannot stand. In this way, the present invention repels external parasites and prevents future infestation, thereby eliminating the need to use chemical anti-parasite products, which carry a risk of side effects.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above descriptions then, it is to be realized that the optimum dimensional relationships for the parts of the invention, to include variations in size, materials, shape, form, function, and manner of operation, assembly and use, are deemed readily apparent and obvious to one skilled in the art, and all equivalent relationships to those illustrated in the drawings and described in the specifications are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A therapeutic magnetic pouch for a pet collar, comprising:
a rectangular pouch having an inner surface that is coextensive with an outer surface, a top portion, a bottom portion, a first side, and a second side;
a central pocket that extends between said first side and said second side;
said top portion having a first fastener;
said bottom portion having a second fastener;
wherein said top portion and said bottom portion can be overlapped so as to form a loop by securing said first fastener and said second fastener;
a plurality of magnets disposed within said central pocket.

2) The therapeutic magnetic pouch for a pet collar of claim 1, wherein each of said first fastener and said second fastener is a hook fastener or a loop fastener.

3) The therapeutic magnetic pouch for a pet collar of claim 1, wherein a north-pole of said plurality of magnets face said outer surface of said pouch.

4) The therapeutic magnetic pouch for a pet collar of claim 1, wherein said outer surface comprises a loop fastener at said bottom portion;
said inner surface comprises a hook fastener at said top portion.

5) The therapeutic magnetic pouch for a pet collar of claim 1, wherein said pouch further comprises a first and a second horizontal rows of stitches to form said central pocket.

6) The therapeutic magnetic pouch for a pet collar of claim 1, wherein said pouch is composed of nylon.

7) The therapeutic magnetic pouch for a pet collar of claim 1, wherein said magnets comprise a disk shape.

8) The therapeutic magnetic pouch for a pet collar of claim 1, wherein said magnets are arranged in a linear configuration.

9) The therapeutic magnetic pouch for a pet collar of claim 1, wherein said magnets are neodymium magnets.

10) The therapeutic magnetic pouch for a pet collar of claim 1, wherein said central pocket is disposed at substantial midpoint between said top portion and said bottom portion.

11) A therapeutic magnetic pouch for a pet collar comprising:
a central pouch having an interior volume in which one or more magnets are positioned;
an upper panel disposed on the upper end of said central pouch;
a lower panel disposed on the lower end of said central pouch;
a first fastener disposed on said upper panel;
a second fastener disposed on said lower panel;
wherein said upper panel and said lower panel can be overlapped so as to form a loop by securing said first fastener to said second fastener.

12) The therapeutic magnetic pouch for a pet collar of claim 11, wherein each of said first fastener and said second fastener is a hook fastener or a loop fastener.
